(19) United States
(12) Patent Application Publication
(10) Pub. No.: US 2021/0204905 A1
(43) Pub. Date: Jul. 8, 2021

(54) ULTRASOUND DIAGNOSTIC APPARATUS AND PULSE SIGNAL TRANSMITTER

(71) Applicant: TERUMO KABUSHIKI KAISHA, Tokyo (JP)

(72) Inventors: Mitsutoshi YAEGASHI, Aomori-shi (JP); Itaru OOKUBO, Naka-gun Ninomiya-cho (JP); Katsuhiko SHIMIZU, Fujinomiya-shi (JP)

(73) Assignee: TERUMO KABUSHIKI KAISHA, Tokyo (JP)

(21) Appl. No.: 17/212,453

(22) Filed: Mar. 25, 2021

Related U.S. Application Data
(63) Continuation of application No. PCT/JP2019/037181, filed on Sep. 24, 2019.

(30) Foreign Application Priority Data
Sep. 25, 2018 (JP) 2018-179189

(51) Int. Cl. A61B 8/08; G01S 15/89; A61B 8/00; A61B 8/12

(52) U.S. Cl. CPC A61B 8/0883; G01S 15/899; A61B 8/445; A61B 8/56; A61B 8/12; G01S 15/8915

(57) ABSTRACT

An ultrasound image generating apparatus is disclosed, which includes a signal output unit configured to be connected to an ultrasound transducer that transmits ultrasound based on a drive signal toward a subject and generates a detection signal based on the ultrasound reflected from the subject, and the signal output unit is configured to output the drive signal to the ultrasound transducer. The signal output unit includes a plurality of pulsers that each output a pulse-like drive signal, and are connected in parallel to the ultrasound transducer, and a buffer that is connected to input sides of the pulsers, and stabilizes a signal to be input into each of the pulsers.

### CROSS-REFERENCES TO RELATED APPLICATIONS

This application is a continuation of International Application No. PCT/JP2019/037181 filed on Sep. 24, 2019, which claims priority to Japanese Patent Application No. 2018-179189 filed on Sep. 25, 2018, the entire content of both of which is incorporated herein by reference.

### FIELD OF THE INVENTION

The present disclosure generally relates to an ultrasound diagnostic apparatus and a pulse signal transmitter.

### BACKGROUND DISCUSSION

Conventionally, a medical appliance is inserted into an organ such as a heart or a blood vessel (hereinafter, described as appropriate as “organ or the like”) to treat the organ or the like. Such treatment is performed by obtaining a state of the organ or the like using a three-dimensional image. For example, Japanese Patent Application Publication No. 2016-64074 discloses an ultrasound diagnostic apparatus that generates a three-dimensional image of an organ or the like.

In order to subject an organ or the like to suitable treatment, the ultrasound diagnostic apparatus that can observe an inside of the organ or the like has been required.

### SUMMARY

It would be desirable to have an ultrasound diagnostic apparatus and a pulse signal transmitter that can increase an observable region inside of the organ or the like.

In accordance with an aspect, an ultrasound image generating apparatus is disclosed that includes a signal output unit capable of being connected to an ultrasound transducer that transmits ultrasound based on a drive signal toward a subject and generates a detection signal based on the ultrasound reflected from the subject, and the signal output unit outputting the drive signal to the ultrasound transducer, in which the signal output unit includes a plurality of pulsers that each output a pulse-like drive signal, and are connected in parallel to the ultrasound transducer, and a buffer that is connected to input sides of the pulsers, and stabilizes a signal to be input into each of the pulsers.

As one embodiment of the present disclosure, the pulser includes a switching element, and generates the drive signal by controlling an on and an off of the switching element.

As one embodiment of the present disclosure, the pulser includes at least a first pulser and a second pulser, the first pulser outputs, as the drive signal, a first pulse train in which a positive pulse and a negative pulse are arranged in a predetermined pattern to a first end of the ultrasound transducer, and the second pulser outputs, as the drive signal, a second pulse train in which the pulses included in the first pulse train are replaced with pulses with reversed signs to a second end of the ultrasound transducer.

As one embodiment of the present disclosure, the ultrasound image generating apparatus is further provided with a drive device configured to be connected to a shaft that is interlocked with the ultrasound transducer, and a control device configured to control the drive device, in which the drive device is provided with the signal output unit, a signal acquisition unit configured to acquire the detection signal from the ultrasound transducer, and a drive unit configure to drive the shaft.

As one embodiment of the present disclosure, in the ultrasound image generating, the control device is configured to generate a diagnostic image based on the detection signal by synchronizing timing at which the signal output unit outputs the drive signal with timing at which the signal acquisition unit acquires the detection signal, based on a trigger signal that is generated in response to the timing at which the drive signal is output.

In accordance with another aspect, a pulse signal transmitter includes a signal output unit for outputting a pulse signal; and a control unit configured to control the signal output unit, in which the signal output unit includes a plurality of pulsers that are connected in parallel to an output destination of the pulse signal, and a buffer that is connected to input sides of the pulsers, and stabilizes a control signal to be input into each of the pulsers.

In accordance with a further aspect, a method is disclosed for intra-atrial imaging, the method comprising: inserting a catheter into a blood vessel of a subject, the catheter including an ultrasound transducer, the ultrasound transducer connected to a signal output unit, the signal output unit including a plurality of pulsers that each output a pulse-like drive signal and are connected in parallel to the ultrasound transducer, the signal output unit further including a buffer that is connected to input sides of the plurality of pulsers and stabilizes a signal being input into each of the plurality of pulsers; and transmitting ultrasound toward the subject based on a drive signal and generating a detection signal based on the ultrasound reflected from the subject.

The ultrasound image generating apparatus and the pulse signal transmitter according to the present disclosure can increase an observable region.

### BRIEF DESCRIPTION OF THE DRAWINGS

FIG. 1 is a block diagram illustrating a configuration example of an ultrasound diagnostic apparatus according to an exemplary embodiment.

FIG. 2 is a perspective view illustrating a configuration example of the ultrasound diagnostic apparatus according to the exemplary embodiment.

FIG. 3 is a cross-sectional view illustrating a configuration example of an ultrasound inspector that is accommodated in a catheter.

FIG. 4 is a view illustrating one example of a state where the catheter is inserted into an inside of a heart.

FIG. 5 is a block diagram illustrating one example of a configuration of a signal output unit.

FIG. 6 is a graph illustrating one example of a drive signal that is represented as a voltage waveform.

FIG. 7 is a block diagram illustrating a configuration example of a signal output unit that is provided with a plurality of pulsers.

FIG. 8A is a graph illustrating an example (output from a first pulser) of a drive signal that is represented as a voltage waveform including positive-negative reversed pulses.

FIG. 8B is a graph illustrating an example (output from a second pulser) of a drive signal that is represented as a voltage waveform including positive-negative reversed pulses.

### DETAILED DESCRIPTION

Set forth below with reference to the accompanying drawings is a detailed description of embodiments of an ultrasound diagnostic apparatus or ultrasound image generating apparatus and a pulse signal transmitter representing examples of the inventive ultrasound diagnostic apparatus and the pulse signal transmitter. Note that since embodiments described below are preferred specific examples of the present disclosure, although various technically preferable limitations are given, the scope of the present disclosure is not limited to the embodiments unless otherwise specified in the following descriptions. In the respective drawings, the components indicated by the same sign are the same or similar.

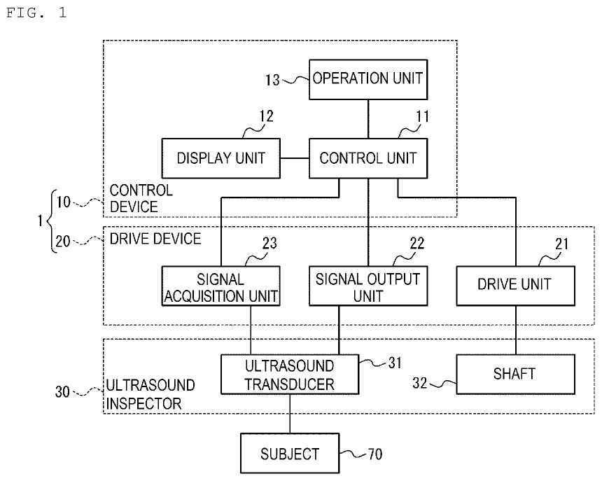

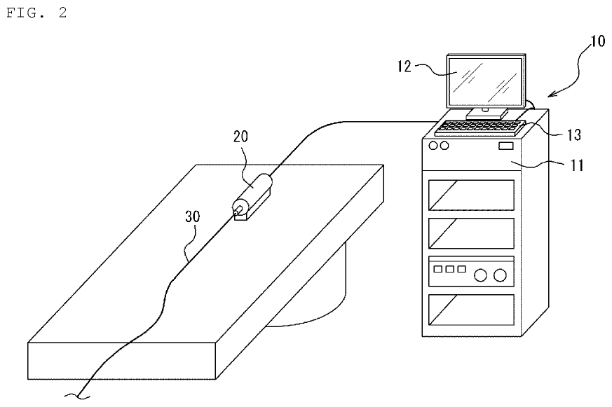

As illustrated in FIGS. 1 and 2, an ultrasound diagnostic apparatus (or ultrasound image generating apparatus) 1 according to the present exemplary embodiment is provided with a control device 10 and a drive device 20. The ultrasound diagnostic apparatus 1 is capable of being connected to an ultrasound inspector 30 via the drive device 20. Hereinafter, the embodiment in which the ultrasound diagnostic apparatus 1 is connected to the ultrasound inspector 30 will be described. The ultrasound inspector 30 transmits ultrasound to a subject 70, and acquires a detection signal based on the ultrasound to be reflected from the subject 70. The detection signal includes information related to the subject 70. The ultrasound diagnostic apparatus 1 acquires a detection signal of the ultrasound inspector 30.

The control device 10 outputs a trigger signal to the drive device 20. The drive device 20 causes, at timing when having acquired the trigger signal, the ultrasound inspector 30 to acquire a detection signal. The control device 10 acquires the detection signal of the ultrasound inspector 30 through the drive device 20. The control device 10 synchronizes the trigger signal output by the control device 10 with the detection signal acquired from the ultrasound inspector 30, and generates a diagnostic image of the subject 70 on the basis of the detection signal.

The trigger signal may be output from the control device 10 to the ultrasound inspector 30. The trigger signal may be output from the drive device 20. When the drive device 20 outputs a trigger signal, the control device 10 synchronizes the trigger signal acquired from the drive device 20 with the detection signal acquired from the ultrasound inspector 30, and generates a diagnostic image of the subject 70 on the basis of the detection signal.

The control device 10 can include a control unit 11, a display unit 12, and an operation unit 13. The drive device 20 can include a drive unit 21, a signal output unit 22, and a signal acquisition unit 23. The drive device 20 is also referred to as a motor drive unit (MDU).

The control device 10 can include the control unit 11, the display unit 12, the operation unit 13, the signal output unit 22, and the signal acquisition unit 23. In accordance with an embodiment, the drive device 20 is provided with the drive unit 21.

The control unit 11 controls the respective constituent units of the control device 10, and the respective constituent units of the drive device 20. The control unit 11 may execute a specified function by reading a specified program. The control unit 11 may include, for example, a processor. The control unit 11 may include a storage unit that stores in the control unit 11 various information and programs. The storage unit may include, for example, a semiconductor memory. The storage unit may be configured separately from the control unit 11. The control unit 11 may also output a trigger signal.

The display unit 12 displays information generated by the control unit 11. The display unit 12 may display a diagnostic image, and may display information related to an operation of the ultrasound diagnostic apparatus 1. The display unit 12 may include, for example, a display device such as a liquid crystal display or an organic electro-luminescence (EL) display.

The operation unit 13 receives an input such as information or an instruction by an operator, and outputs the information and the instruction to the control unit 11. The operation unit 13 may include, for example, an input device such as a keyboard, a mouse, or a touch panel. When the operation unit 13 includes a touch panel, the touch panel may be configured integrally with the display unit 12.

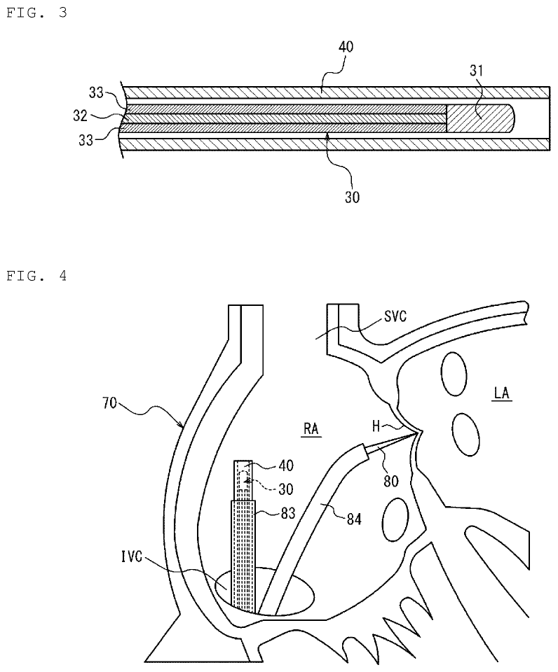

As illustrated in FIG. 3, the ultrasound inspector 30 is accommodated in a catheter 40. An operator such as a health care worker inserts the catheter 40 into a blood vessel, whereby the ultrasound inspector 30 reaches an inside of an organ such as a heart or the blood vessel. An organ such as a heart or a blood vessel is also referred to as “organ or the like” hereinafter. The operator operates the ultrasound inspector 30 at a hand side (i.e., proximal side), and observes an inside of the organ or the like. The ultrasound inspector 30 includes an end portion (or distal portion) at a side (distal side) where the ultrasound inspector 30 is inserted into the inside of the organ or the like, and an end portion (proximal portion) at the hand side where the ultrasound inspector 30 is operated. The end portion at the side where the ultrasound inspector 30 is inserted is also referred to as a distal portion. The end portion at the hand side where the ultrasound inspector 30 is operated is also referred to as a proximal portion. The ultrasound inspector 30 and the catheter 40 may be integrally configured, for example, as an ultrasound catheter.

The ultrasound inspector 30 is provided with an ultrasound transducer 31, a shaft 32, and a tube 33. The ultrasound transducer 31 transmits ultrasound (i.e., ultrasonic waves) toward the subject 70, and receives the ultrasound (ultrasonic waves) reflected from the subject 70. The shaft 32 can be a linear member having flexibility. The shaft 32 is interlock with the ultrasound transducer 31 at a distal portion of the shaft 32, and interlocked with the drive unit 21 at a proximal portion of the shaft 32. The tube 33 is a tubular member having flexibility, and covers a circumferential direction of the shaft 32. The tube 33 is in close contact with the shaft 32, and thus is capable of sliding an extending direction relative to the catheter 40 without hindering the rotation and the movement of the shaft 32. Moreover, in order to rather easily transmit a hand-pushing force on a proximal side of the ultrasound inspector 30 to a distal side of the ultrasound inspector 30, a proximal portion of the tube 33 can be harder than a distal portion of the tube 33.

For example, as illustrated in FIG. 4, the catheter 40 may be inserted into an inside of a heart as the subject 70. The catheter 40 is inserted into an inside of a right atrium RA through a first sheath 83 that is inserted into the right atrium RA via an inferior vena cava IVC. The catheter 40 may be inserted up to a superior vena cava SVC. A Brockenbrough needle 80 can be inserted into the inside of the right atrium RA through a second sheath 84 that is inserted into the right atrium RA via the inferior vena cava IVC. The Brockenbrough needle 80 can be used to penetrate through a foramen ovale H separating the right atrium RA and a left atrium LA, and to open the left atrium LA from the right atrium RA. In accordance with an aspect, the ultrasound inspector 30 outputs a detection signal related to the Brockenbrough needle 80 and a state of an inner wall of the left atrium LA, to the signal acquisition unit 23. The control unit 11 generates a diagnostic image for grasping a position of the Brockenbrough needle 80 and a state of the inner wall of the left atrium LA by the operator, on the basis of the detection signal.

The drive unit 21 drives the shaft 32 to move the ultrasound transducer 31 that is interlocked with the distal portion of the shaft 32 along the extending direction of the catheter 40, and to rotate the ultrasound transducer 31 along the circumferential direction of the catheter 40. The drive unit 21 may include a driving mechanism such as a motor. The drive unit 21 may be provided with an interface that receives an operation input by the operator. The operator of the ultrasound diagnostic apparatus 1 can obtain a diagnostic image at a desired position of the subject 70 by controlling a position and a posture of the ultrasound transducer 31 with the drive unit 21.

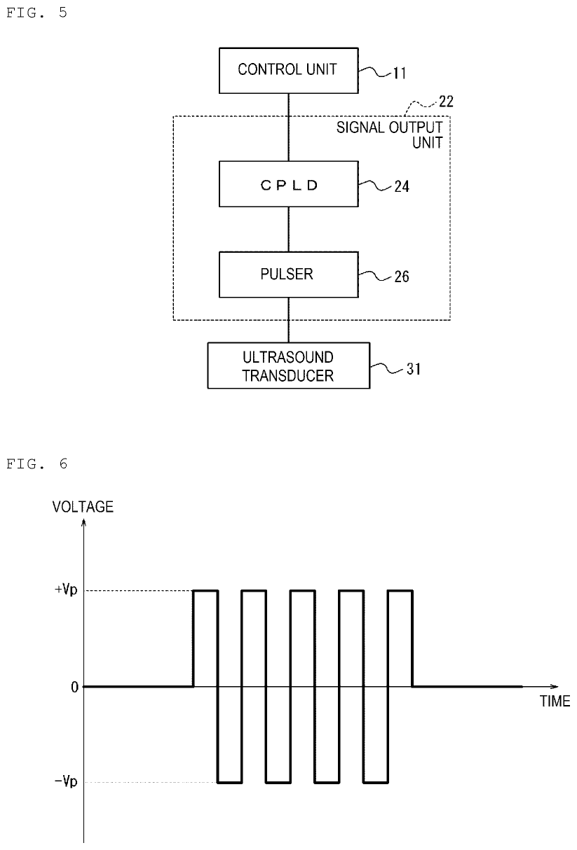

The signal output unit 22 outputs a signal for applying a voltage to the ultrasound transducer 31. A signal for applying a voltage to the ultrasound transducer 31 is also referred to as a drive signal. The signal output unit 22 is electrically connected to the ultrasound transducer 31 with a signal line that is provided inside the shaft 32. As illustrated in FIG. 5, the signal output unit 22 can be provided with a programmable logic device, for example, a complex programmable logic device (CPLD) 24, and a pulser 26. For example, the CPLD 24 outputs a plurality of control signals (for example, digital signals) for applying a voltage to the ultrasound transducer 31.

In accordance with an embodiment, the pulser 26 outputs a pulse signal of a rectangular wave. The voltage at the pulse signal is larger than the voltage at the control signal. The pulser 26 includes a terminal through which a control signal is acquired from the CPLD 24. The terminal through which a control signal is acquired from the CPLD 24 is also referred to as a signal input terminal (for example, a digital signal input terminal). The pulser 26 includes a switching element, and controls on/off of the switching element on the basis of the control signal acquired from the CPLD 24. The switching element may include, for example, a semiconductor element such as a metal oxide semiconductor field effect transistor (MOSFET). The pulser 26 may transition, by controlling on/off of the switching element, to any of an off state in which no voltage is output, a first state in which a first voltage is output, and a second state in which a second voltage is output. The first voltage may be a positive voltage. The second voltage may be a negative voltage. An absolute value of the first voltage and an absolute value of second voltage may be equal to or different from each other. The pulser 26 may include a constant voltage circuit that generates the first voltage and the second voltage. The pulser 26 may be connected to an external power supply circuit, and may acquire the first voltage and the second voltage from the external power supply circuit.

When the pulser 26 applies a voltage to the ultrasound transducer 31, a current flows from the pulser 26 to the ultrasound transducer 31. For example, when the pulser 26 applies the first voltage to the ultrasound transducer 31, a current based on the application of the first voltage flows to the ultrasound transducer 31. The ultrasound transducer 31 may be of a piezoelectric type, such as piezoelectric ceramics. When the ultrasound transducer 31 is of a piezoelectric type, the ultrasound transducer 31 mainly acts as a capacitive load with respect to the application of the voltage. As the relative (physical) size of the ultrasound transducer 31 becomes larger, the current flowing through the ultrasound transducer 31 becomes relatively large.

The pulser 26 can include a plurality of digital signal input terminals. The digital signal input terminals may include a first signal input terminal and a second signal input terminal. The pulser 26 may transition to the first state when having acquired a control signal at the first signal input terminal. The pulser 26 may transition to the second state when having acquired a control signal at the second signal input terminal. The pulser 26 may transition to the off state when having acquired a control signal at neither of the first signal input terminal nor the second signal input terminal. The pulser 26 may transition to the first state, the second state, or the off state in accordance with a combination of High and Low (i.e., high voltage and low voltage) of each of the first signal input terminal and the second signal input terminal. The signal input terminal may further include an enable terminal through which an enable signal is acquired.

The CPLD 24 generates a control signal corresponding to each signal input terminal, which causes the pulser 26 to transition to a desired state, and outputs the control signal to each signal input terminal. The CPLD 24 may output a control signal to the pulser 26 at the timing when having acquired the trigger signal from the control unit 11. The CPLD 24 controls the timing when outputting a control signal to the pulser 26, thereby controlling the waveform of the voltage that is output as a drive signal by the pulser 26. For example, the CPLD 24 may control the timing when outputting a control signal so as to cause the pulser 26 to output a burst wave as a drive signal. The burst wave is a signal including a period during which a periodic waveform such as a pulse or a sine wave is continuously output and a period during which no waveform is output. The drive signal may include, for example, as exemplified in FIG. 6, a pulse train in which five positive pulses and four negative pulses are alternately arranged. The drive signal including such a pulse train is also referred to as a burst wave of 4.5 waves. In other words, the pulser 26 may output a pulse-like drive signal. In FIG. 6, a horizontal axis and a longitudinal axis respectively represent time and voltage. Positive and negative amplitudes of the drive signal are respectively represented as +Vp and −Vp. The drive signal may be a burst wave of two waves including two pairs of positive and negative pulses. The number of pulses included in the drive signal is not limited to these. For example, as the number of pulses included in the drive signal is reduced, the time during which ultrasound is transmitted from the ultrasound transducer 31 becomes shorter, thereby increasing the resolution of the diagnostic image. Moreover, as the number of pulses included in the drive signal is increased, the time during which ultrasound is transmitted from the ultrasound transducer 31 becomes longer, thereby increasing intensity of the detection signal.

The signal acquisition unit 23 acquires a detection signal from the ultrasound transducer 31. The ultrasound transducer 31 outputs a result of detecting ultrasound reflected from the subject 70, as a detection signal, to the signal acquisition unit 23. The signal acquisition unit 23 may include an amplifier such as a preamplifier that amplifies a signal. The signal acquisition unit 23 may amplify a detection signal acquired from the ultrasound transducer 31 by the amplifier, and output the amplified detection signal to the control unit 11.

In accordance with an aspect, the drive device 20 can two-dimensionally scan the ultrasound that is transmitted to the subject 70 by causing the ultrasound transducer 31 to transmit the ultrasound by the signal output unit 22 while controlling the position and the angle of the ultrasound transducer 31 by the drive unit 21. The control unit 11 can generate a two-dimensional diagnostic image by synchronizing the detection signal that is acquired from the signal acquisition unit 23 with information related to the control of the position and the angle of the ultrasound transducer 31 by the drive unit 21.

The drive signal that is output from the signal output unit 22 is attenuated while propagating through the signal line. As the intensity of the drive signal that propagates to the ultrasound transducer 31 is larger, the intensity of the ultrasound that is transmitted by the ultrasound transducer 31 is larger. As the intensity of the ultrasound is larger, an observation range of the subject 70 that is included in the diagnostic image becomes wider.

In the ultrasound diagnostic apparatus 1 according to the present embodiment, the signal output unit 22 is included in the drive device 20. Meanwhile, in an apparatus according to a comparative example 1, the signal output unit 22 is included in the control device 10. The ultrasound diagnostic apparatus 1 according to the present embodiment can shorten the signal line from the signal output unit 22 to the ultrasound transducer 31, and can reduce the attenuation amount during when the drive signal propagates from the signal output unit 22 to the ultrasound transducer 31, as compared with the comparative example 1. As a result, the ultrasound diagnostic apparatus 1 according to the present embodiment can increase the intensity of the ultrasound that is transmitted from the ultrasound transducer 31, and can widen the observation range of the subject 70 that is included in the diagnostic image. Thus, the ultrasound diagnostic apparatus 1 according to the present embodiment can generate a diagnostic image for observing a wide range because the drive device 20 is provided with the signal output unit 22.

In the ultrasound diagnostic apparatus 1 according to the present embodiment, the control device 10 and the drive device 20 operate in synchronization by the transmission and reception of a trigger signal. In this manner, even if the signal output unit 22 that is an output source of the drive signal is not included in the control device 10, the control device 10 can synchronize the timing at which a drive signal is output with the timing at which a detection signal is acquired, and generate a diagnostic image.

In the ultrasound diagnostic apparatus 1 according to the present embodiment, the signal output unit 22 generates and outputs, as a drive signal of the ultrasound transducer 31, a pulse of a rectangular wave based on on/off of the switching element, by the pulser 26. Meanwhile, in an apparatus according to a comparative example 2, the signal output unit 22 outputs an analog signal including a sine wave or the like. A circuit that generates an analog signal is likely to be larger in physical size than the pulser 26. In addition, a circuit that amplifies the analog signal is necessary. Therefore, as compared with the signal output unit 22 in the apparatus according to the comparative example 2, the signal output unit 22 in the ultrasound diagnostic apparatus 1 according to the present embodiment is likely to be downsized (i.e., physically smaller). In other words, in the ultrasound diagnostic apparatus 1 according to the present embodiment, the signal output unit 22 can be downsized because the signal output unit 22 is provided with the pulser 26. The drive device 20 is disposed near a subject so as to allow the operator to operate near the subject. Moreover, the drive device 20 is required to be downsized so as to be rather easily handled by the operator. With the ultrasound diagnostic apparatus 1 according to the present embodiment, while the drive device 20 is downsized, the signal output unit 22 is accommodated inside the drive device 20. As a result, the ultrasound diagnostic apparatus 1 according to the present embodiment can reduce the attenuation amount of the drive signal, and generate a diagnostic image for observing a wide range.

For example, when the subject 70 is an organ such as a heart, the generation of a diagnostic image in a wider range is required. The ultrasound diagnostic apparatus 1 causes the ultrasound that is transmitted from the ultrasound inspector 30 to reach a wider range, for the generating of a diagnostic image in a wider range. The ultrasound transducer 31 may be made relatively larger in physical size in order to cause the ultrasound to reach in the wider range. As the ultrasound transducer 31 becomes larger, the current necessary for driving the ultrasound transducer 31 becomes larger. The current necessary for driving the ultrasound transducer 31 exceeds, when the ultrasound transducer 31 becomes larger, the current capable of being stably output from one pulser 26 in some cases. In other words, the ultrasound transducer 31 cannot be stably driven only by the drive signal that is output from one pulser 26 in some cases.

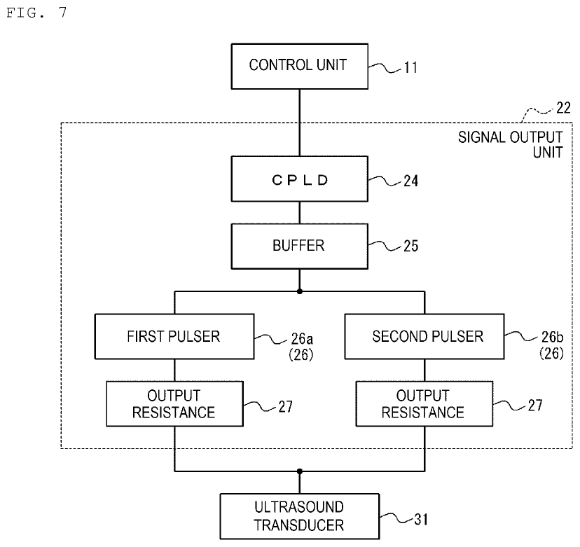

As illustrated in FIG. 7, the signal output unit 22 may be provided with a plurality of the pulsers 26. The plurality of the pulsers 26 may include a first pulser 26a and a second pulser 26b. The first pulser 26a and the second pulser 26b may be connected in parallel to the ultrasound transducer 31. In this case, while the voltage that is applied from each pulser 26 to the ultrasound transducer 31 is maintained, the current flowing from each pulser 26 to the ultrasound transducer 31 can be reduced. In this manner, the current flowing in the ultrasound transducer 31 by each pulser 26 becomes a value lower than the current capable of being stably output by each pulser 26. As a result, even when the size of the ultrasound transducer 31 becomes relatively large, the signal output unit 22 can stably output the current to the ultrasound transducer 31. Note that, the control unit 11 and the signal output unit 22 configure a pulse signal transmitter for outputting a drive signal (pulse signal) to the ultrasound transducer 31 that is an output destination.

The signal output unit 22 may be further provided with a buffer 25 at input sides of the respective pulsers 26. The buffer 25 can correct an input voltage lowered by electric resistance of the circuit, and output the corrected input voltage. Therefore, the buffer 25 branches and equally outputs a control signal into the respective pulsers 26 to stabilize the control signal that is output from the CPLD 24. The transmission time of the control signal from the buffer 25 to the respective pulsers 26 is approximately the same. In this manner, the timing at which the respective pulsers 26 output drive signals is likely to be synchronized. The transmission time of the control signal from the buffer 25 to the respective pulsers 26 is approximately the same, for example, so that the wiring lengths from the buffer 25 to the respective pulsers 26 may be made to be approximately the same. Moreover, a delay circuit may be connected between the buffer 25 and the respective pulsers 26. Moreover, the signal output unit 22 may be provided with the buffers 25 respectively with respect to the respective pulsers 26. Even if the input current to the pulser 26 is insufficient, the control signal that is output from the CPLD 24 can be stabilized.

In accordance with an aspect, the signal output unit 22 may be further provided with an output resistance 27 at an output side of each pulser 26. The output resistance 27 reduces a difference in the magnitude of the currents that are output by the respective pulsers 26. Therefore, the output resistance 27 can improve the uniformity of the currents caused to flow by the respective pulsers 26 that are connected in parallel to the ultrasound transducer 31. In this manner, in any of the plurality of the pulsers 26, the current that is caused to flow in the ultrasound transducer 31 is lower than the current capable of being stably output. As a result, the stability of the signal output unit 22 can be improved. The resistance value by the output resistance 27 may be set as appropriate.

The ultrasound diagnostic apparatus 1 according to the present embodiment, which is provided with the plurality of the pulsers 26, can stably drive the ultrasound transducer 31, even when the size of the ultrasound transducer 31 becomes relatively large. As a result, a diagnostic image in a wider range can be generated.

The ultrasound transducer 31 may be connected to a reference potential point at a first end, and may be connected to the pulser 26 at a second end. In this case, the voltage to be applied to the ultrasound transducer 31 corresponds to a potential difference between a potential of a drive signal that is applied from the pulser 26 and a reference potential. The ultrasound transducer 31 may be connected to the second pulser 26b at the second end while being connected to the first pulser 26a at the first end. In this case, the voltage to be applied to the ultrasound transducer 31 corresponds to a potential difference between a potential of a drive signal that is applied from the first pulser 26a and a potential of a drive signal that is applied from the second pulser 26b.

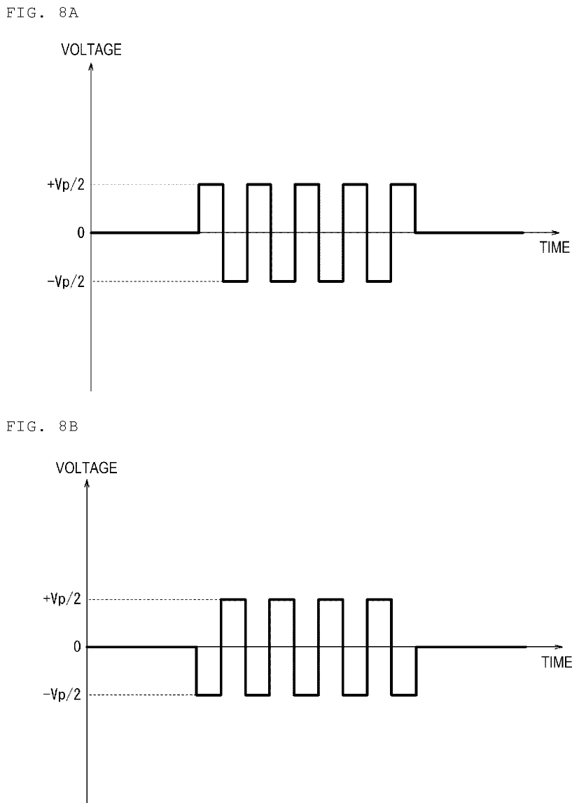

When the first pulser 26a and the second pulser 26b are respectively connected to the first end and the second end of the ultrasound transducer 31, the first pulser 26a and the second pulser 26b may respectively output pulses with the reversed positive and negative signs. For example, the first pulser 26a and the second pulser 26b may respectively output drive signals of voltage waveforms exemplified in FIGS. 8A and 8B. In FIGS. 8A and 8B, each horizontal axis and each longitudinal axis respectively represent time and voltage. The voltage waveform illustrated in FIG. 8A includes a first pulse train in which five positive pulses and four negative pulses are alternately arranged. Therefore, it can be said that positive pulses and negative pulses are arranged in a predetermined pattern in the first pulse train. The voltage waveform illustrated in FIG. 8B includes a second pulse train in which five negative pulses and four positive pulses are alternately arranged. Therefore, it can be said that the pulses included in the first pulse train are replaced with the pulses with the reversed signs in the second pulse train. Positive and negative amplitudes of the pulses illustrated in FIGS. 8A and 8B are respectively represented as +Vp/2 and −Vp/2.

When the voltage based on the voltage waveforms exemplified in FIGS. 8A and 8B is input into the both ends of the ultrasound transducer 31, the voltage that is applied to the both ends of the ultrasound transducer 31 corresponds to a potential difference between the voltage waveform in FIG. 8A and the voltage waveform in FIG. 8B, and is represented as the voltage waveform illustrated in FIG. 6. An absolute value of the amplitude of the voltage waveform illustrated in FIG. 6 is represented as Vp. Meanwhile, an absolute value of the amplitude of each of the voltage waveforms illustrated in FIGS. 8A and 8B is Vp/2. Therefore, the voltage waveform illustrated in FIG. 6 is generated as a synthesized result of the voltage waveforms illustrated in FIGS. 8A and 8B.

In this manner, outputs from the respective pulsers 26 are synthesized, so that the voltage at the synthesized output becomes relatively larger than the voltage that is output from each pulser 26. In other words, the output from each pulser 26 may be reduced. As a result, the power supply voltage to be supplied to each pulser 26 can be lowered.

The ultrasound diagnostic apparatus 1 according to the present embodiment is provided with at least the two pulsers 26 that output pulses with the reversed positive and negative signs, thereby making it possible to lower the power supply voltage. As a result, the device is further downsized.

The present disclosure is not limited to the configuration specified in the above-mentioned embodiment, but various modifications are possible without deviating from the scope of the present disclosure. For example, the respective constituent units, the functions included in the respective steps, and the like can be reconstructed unless causing the logical contradictions, and a plurality of constituent units or steps can be combined as one, or can be divided.

Moreover, the pulse signal transmitter according to the present disclosure that is provided with the control unit 11 and the signal output unit 22 can be applied to apparatuses other than the ultrasound diagnostic apparatus 1. For example, the pulse signal transmitter can be applied to an ablation apparatus including an electrode that receives a pulse signal from the signal output unit 22 and causes the current to flow in a biological tissue, in place of the ultrasound transducer 31. Also, in such an ablation apparatus, the signal output unit 22 is provided with the plurality of the pulsers 26 that are connected in parallel to the electrode that is an output destination of a pulse signal, so that it is possible to reduce the current flowing from the respective pulsers 26 to the electrode while maintaining the voltage to be applied to the electrode from the respective pulsers 26. In this manner, the current that is caused to flow in the electrode by each pulser 26 becomes a value lower than the current capable of being stably output by each pulser 26. As a result, even when a relatively high voltage is applied to the electrode, the signal output unit 22 can stably output the current to the electrode. Note that, in such an ablation apparatus, the signal output unit 22 is incorporated into the control device 10, and the drive device 20 is omitted.

The detailed description above describes embodiments of an ultrasound diagnostic apparatus and a pulse signal transmitter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An ultrasound image generating apparatus comprising:
a signal output unit configured to be connected to an ultrasound transducer, the signal output unit configured to output a drive signal to the ultrasound transducer, and wherein the ultrasound transducer is configured to transmit ultrasound toward a subject based on the drive signal, and to generate a detection signal based on the ultrasound reflected from the subject; and
wherein the signal output unit includes a plurality of pulsers that each output a pulse-like drive signal, and are connected in parallel to the ultrasound transducer, and a buffer that is connected to input sides of the plurality of pulsers, and configured to stabilize a signal to be input into each of the plurality of pulsers.

2. The ultrasound image generating apparatus according to claim 1, wherein the plurality of pulsers each includes a switching element, and wherein the plurality of pulser is configured to generate the drive signal by controlling an on and an off of the switching element.

3. The ultrasound image generating apparatus according to claim 1, Wherein the plurality of pulsers includes at least a first pulser and a second pulser;
the first pulser is configured to output, as the drive signal, a first pulse train in which a positive pulse and a negative pulse are arranged in a predetermined pattern to a first end of the ultrasound transducer; and
the second pulser is configured to output, as the drive signal, a second pulse train in which the pulses included in the first pulse train are replaced with pulses with reversed signs to a second end of the ultrasound transducer.

4. The ultrasound image generating apparatus according to claim 1, further comprising:
a drive device configured to be connected to a shaft that is interlocked with the ultrasound transducer, and a control device configured to control the drive device; and
the drive device being provided with the signal output unit, a signal acquisition unit configured to acquire the detection signal from the ultrasound transducer, and a drive unit configured to drive the shaft.

5. The ultrasound image generating apparatus according to claim 4, wherein the control device is configured to generate a diagnostic image based on the detection signal by synchronizing timing at which the signal output unit outputs the drive signal with timing at which the signal acquisition unit acquires the detection signal, based on a trigger signal that is generated in response to the timing at which the drive signal is output.

6. The ultrasound image generating apparatus according to claim 1, wherein the signal output unit further includes a programmable logic device configured to output digital signals for applying a voltage to the ultrasound transducer.

7. The ultrasound image generating apparatus according to claim 1, wherein the ultrasound transducer is part of an ultrasound inspector, the ultrasound inspector including a tubular member and a shaft housed within the tubular member and interlocked with a drive shaft on a proximal end and the ultrasound transducer on a distal end.

8. The ultrasound image generating apparatus according to claim 7, wherein the ultrasound inspector is housed within a catheter.

9. The ultrasound image generating apparatus according to claim 1, wherein the plurality of pulsers output a pulse signal of a rectangular wave.

10. A pulse signal transmitter comprising:
a signal output unit configured to output a pulse signal;
a control unit configured to control the signal output unit; and
wherein the signal output unit includes a plurality of pulsers that are connected in parallel to an output destination of the pulse signal, and a buffer that is connected to input sides of the plurality of pulsers, and configured to stabilize a control signal to be input into each of the plurality of pulsers.

11. The pulse signal transmitter according to claim 10, wherein the plurality of pulsers each includes a switching element, and wherein the plurality of pulser is configured to generate the drive signal by controlling an on and an off of the switching element.

12. The pulse signal transmitter according to claim 10,
Wherein the plurality of pulsers includes at least a first pulser and a second pulser;
the first pulser is configured to output, as the drive signal, a first pulse train in which a positive pulse and a negative pulse are arranged in a predetermined pattern; and
the second pulser is configured to output, as the drive signal, a second pulse train in which the pulses included in the first pulse train are replaced with pulses with reversed signs.

13. The pulse signal transmitter according to claim 10, further comprising:
a programmable logic device configured to output control signals to the input sides of the plurality of pulsers, the control signals configured to transition the plurality of pulsers to a desired state.

14. The pulse signal transmitter according to claim 10, further comprising:
an ablation apparatus including an electrode configured to receive the pulse signal from the signal output unit and configured to cause a current to flow in a biological tissue.

15. The pulse signal transmitter according to claim 14, wherein the plurality of the pulsers are connected in parallel to the electrode.

16. The pulse signal transmitter according to claim 15, wherein the signal output unit is incorporated into the control device.

17. A method for intra-atrial imaging, the method comprising:
inserting a catheter into a blood vessel of a subject, the catheter including an ultrasound transducer, the ultrasound transducer connected to a signal output unit, the signal output unit including a plurality of pulsers that each output a pulse-like drive signal and are connected in parallel to the ultrasound transducer, the signal output unit further including a buffer that is connected to input sides of the plurality of pulsers and stabilizes a signal being input into each of the plurality of pulsers; and
transmitting ultrasound toward the subject based on a drive signal and generating a detection signal based on the ultrasound reflected from the subject.

18. The method according to claim 17, further comprising:
inserting the catheter into an inside of a heart of the subject.

19. The method according to claim 18, further comprising:
inserting the catheter into an inside of a right atrium of the heart through a first sheath that is inserted into the right atrium via an inferior vena cava;
inserting a needle into an inside of the right atrium through a second sheath that is inserted into the right atrium via the inferior vena cava;
penetrating the needle through a foramen ovale separating the right atrium and a left atrium and opening the left atrium from the right atrium; and
outputting a detection signal from the ultrasound transducer related to the needle and a state of an inner wall of the left atrium to a signal acquisition unit.

20. The method according to claim 19, further comprising:
generating a diagnostic image for grasping a position of the needle and a state of the inner wall of the left atrium based on the detection signal.
